(19) United States
(12) Patent Application Publication
(10) Pub. No.: US 2010/0028381 A1
(43) Pub. Date: Feb. 4, 2010

(54) FORMULATION FOR DELIVERY OF IMMUNE RESPONSE MODIFIERS

(75) Inventors: Kevin S. Gorski, Newbury Park, CA (US); Ronnie Ortiz, Apple Valley, MN (US); James D. Stoesz, Inver Grove Heights, MN (US); Isidro Angelo E. Zarraga, Millbrae, CA (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, Saint Paul, MN (US)

(21) Appl. No.: 12/304,339

(22) PCT Filed: Jun. 18, 2007
(86) PCT No.: PCT/US2007/071433

§ 371 Date: Oct. 14, 2009

Related U.S. Application Data
(60) Provisional application No. 60/805,147, filed on Jun. 19, 2006.

(51) Int. Cl. A61K 39/12; A61K 39/00; A61K 39/02; A61P 35/00; A61P 37/04; A61P 31/04; A61P 31/12

(52) U.S. Cl. 424/204.1; 424/277.1; 424/234.1

(57) ABSTRACT

The present invention provides pharmaceutical compositions that include an IRM-PEG complex and an antigen, formulated together in a thermoresponsive gel. In another aspect, the present invention also provides a method of eliciting an antigen-specific immune response in a subject. Generally, the method includes administering to the subject a pharmaceutical composition comprising an IRM-PEG complex and an antigen, formulated together in a thermoresponsive gel, in an amount effective to generate an immune response in the subject against the antigen. In yet another aspect, the present invention also provides a method of treating a condition in a subject. Generally, the method includes administering to the subject a pharmaceutical composition comprising an IRM-PEG complex and an antigen, formulated together in a thermoresponsive gel, in an amount effective to ameliorate at least one symptom or clinical sign of the condition.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRM compounds), appear to act through basic immune system mechanisms known as Toll-like receptors (TLRs) to induce selected cytokine biosynthesis, induction of co-stimulatory molecules, and increased antigen-presenting capacity.

They may be useful for treating a wide variety of diseases and conditions. For example, certain IRM compounds may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), auto-immune diseases (e.g., multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929; and International Publication Number WO 2005/079195) and more are still being discovered. Other IRM compounds have higher molecular weights, such as oligonucleotides, including CpGs (see, e.g., U.S. Pat. No. 6,194,388).

Various formulations and dosage forms for delivering IRM compounds have been developed. Formulations include, for example, solutions, suspensions, emulsions, and other mixtures. Dosage forms include, for example, a cream, an ointment, an aerosol formulation, a non-aerosol spray, a gel, a lotion, and the like. Certain formulations may provide a depot effect (see, for example, U.S. Patent Publication No. US2004/026535 1). And certain formulations may include IRM derivatives such as, for example, lipid-modified IRM compounds (International Patent Publication No. WO2005/018555), PEG-ylated IRM compounds (International Patent Publication No. WO2005/110013), or IRM compounds attached to macromolecular supports (U.S. Patent Publication No. US2005/0258698).

In view of the great therapeutic potential for IRM compounds, and despite the important work that has already been done, there is a substantial ongoing need to expand their uses and therapeutic benefits.

### SUMMARY

It has been found that IRM formulations that include an IRM-PEG complex and an antigen formulated together in a thermoresponsive gel can provide improved antigen-specific immunogenicity.

Accordingly, the present invention provides pharmaceutical compositions that include an IRM-PEG complex and an antigen, formulated together in a thermoresponsive gel.

In another aspect, the present invention also provides a method of eliciting an antigen-specific immune response in a subject. Generally, the method includes administering to the subject a pharmaceutical composition comprising an IRM-PEG complex and an antigen, formulated together in a thermoresponsive gel, in an amount effective to generate an immune response in the subject against the antigen.

In yet another aspect, the present invention also provides a method of treating a condition in a subject. Generally, the method includes administering to the subject a pharmaceutical composition comprising an IRM-PEG complex and an antigen, formulated together in a thermoresponsive gel, in an amount effective to ameliorate at least one symptom or clinical sign of the condition.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is a bar graph showing immune response generated by systemic availability of one embodiment of the pharmaceutical compositions of the invention.

FIG. 2 is a bar graph showing a localized antigen-specific immune response generated by one embodiment of the pharmaceutical compositions of the invention.

### DETAILED DESCRIPTION OF ILLUSTRATIVE EMBODIMENTS OF THE INVENTION

The present invention provides pharmaceutical compositions that generally include an IRM-PEG complex and an antigen provided in a thermoresponsive gel. As noted above, certain forms of IRM-PEG complexes are known, as is the formulation of IRM compounds in gel formulations. It has been found that providing an IRM-PEG complex and an antigen formulated together in a thermoresponsive gel can provide benefits that are greater than the sum of the separate benefits provided by IRM-PEG complexes and IRM gel formulations. Generally, compositions that include an IRM-PEG complex and an antigen in a thermoresponsive gel can provide enhanced antigen-specific immunogenicity and reduced systemic side effects. Therefore, the present invention may provide particularly effective compositions for targeted immunotherapy—i.e., for treating certain types of infectious and/or neoplastic conditions.

For example, an IRM-PEG complex and a tumor-specific antigen formulated in a thermoresponsive gel may be administered in the vicinity of a tumor to generate an antigen-specific immune response against the tumor. The therapy enlists the patient's immune system to fight the tumor, which can reduce the need for radiation and/or chemotherapy, each of which can generate undesirable side effects. Because the immune response is antigen-specific, it targets only the tumor cells, thereby minimizing general systemic side effects.

As another example, an IRM-PEG complex and a virus-specific antigen formulated in a thermoresponsive gel may be administered in the vicinity of a tissue infected with a virus (e.g., administering to the liver in a patient having hepatitis). Again, because the patient's immune response is antigen-specific, only the diseased tissues infected with the virus are targeted, thereby minimizing systemic side effects.

Also, the compositions of the invention may be used to treat conditions unrelated to infectious diseases and cancer such as, for example, allergy (ragweed, cedar etc.), Alzheimer's disease (with peptides such as beta-amyloid), and contraception.

The compositions of the invention tend to reduce systemic release of the IRM portion of the composition, further reducing the extent and/or likelihood of side effects. Moreover, an IRM-PEG complex and antigen formulated in a thermoresponsive gel also may induce the immune system more efficiently than, for example, a simple mixture of an IRM-PEG complex and antigen in, for example, an aqueous carrier, thereby generating a stronger immune response to the target tissue (e.g., tumor, infected tissue, etc.) and, once again, reducing the likelihood and/or extent of any side effects.

As used herein, the following terms shall have the indicated meanings:

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR8 agonist) or a particular combination of TLRs (e.g., a TLR 7/8 agonist—an agonist of both TLR7 and TLR8).

“Ameliorate” refers to any reduction in the extent, severity, frequency, and/or likelihood of a symptom or clinical sign characteristic of a particular condition.

“Antigen” and variations thereof refer to any material capable of raising an immune response in a subject challenged with the material. In various embodiments, an antigen may raise a cell-mediated immune response, a humoral immune response, or both. Suitable antigens may be synthetic or occur naturally and, when they occur naturally, may be endogenous (e.g., a self-antigen) or exogenous. Suitable antigenic materials include but are not limited to peptides or polypeptides (including a nucleic acid, at least a portion of which encodes the peptide or polypeptide); lipids; glycolipids; polysaccharides; carbohydrates; polynucleotides; prions; live or inactivated (e.g., attenuated, heat-killed, fixed, irradiated, etc) bacteria, viruses, fungi, or parasites; and bacterial, viral, fungal, protozoal, tumor-derived, or organism-derived immunogens, toxins or toxoids.

“Thermoresponsive gel” and variations thereof refer to compositions that provide a sequestering of active components of a composition with respect to time and/or location. Thus, a thermoresponsive gel may provide for localized—as opposed to systemic—delivery of a pharmaceutical composition. A thermoresponsive gel also may provide delayed release of the active components of a pharmaceutical composition. Delayed released refers to delaying the onset of release rather than extended release, in which the duration of the release period is elongated.

“IRM activity” refers to one or more of the following: activation, clonal expansion of T and B cells specific to an antigen, an increase in T cell effector functions such as cytokine production and killing of infected or transformed cells, and activation of dendritic cells and natural killer cells.

“IRM-PEG complex” and variations thereof (including “PEG-ylated IRM compound”) refers to any complex that includes at least one IRM moiety and at least one PEG moiety.

“Moiety” and variations thereof refer to a portion of a chemical compound that exhibits a particular character such as, for example, a particular biological or chemical function (e.g., immunomodulation and/or target specificity), or a physical property (e.g., size, hydrophilicity or hydrophobicity).

“PEG” and variations thereof refer to polyethylene glycol.

“PEO” and variations thereof refer to polyethylene oxide.

“PLGA” and variations thereof refer to poly(d,l-lactide-co-glycolide).

“PPO” and variations thereof refer to polypropylene oxide.

“Prodrug” refers to a derivative of a drug molecule that can undergo a chemical or enzymatic biotransformation, thereby releasing the active parent drug in the body.

“Selective” and variations thereof refer to having a differential or a non-general impact on biological activity. An agonist that selectively modulates biological activity through a particular TLR may be a TLR-selective agonist. TLR-selectivity may be described with respect to a particular TLR (e.g., TLR8-selective) or with respect to a particular combination of TLRs (e.g., TLR 7/9-selective).

“Sign” or “clinical sign” refers to an objective physical finding relating to a particular condition capable of being found by one other than the patient.

“Symptom” refers to any subjective evidence of disease or of a patient's condition.

As used herein, “a,” “an,” “the,” “at least one,” and “one or more” are used interchangeably. Thus, for example, an IRM-PEG complex comprising “an” IRM moiety can be interpreted to mean that the IRM-PEG complex includes at least one IRM moiety.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

Also herein, the recitations of numerical ranges by endpoints include all numbers subsumed within that range (e.g., 1 to 5 includes 1, 1.5, 2, 2.75, 3, 3.80, 4, 5, etc.).

Generally, the pharmaceutical compositions of the invention include a thermoresponsive gel having as active components an IRM-PEG complex and an antigen.

The IRM-PEG complex includes two moieties: and IRM moiety and a PEG moiety. The IRM moiety may be, or be derived from any suitable IRM compound. The IRM moiety possesses or, in the case of certain embodiments described below in which the IRM moiety is in the form of an IRM prodrug, has the potential to possess IRM activity.

IRM compounds generally include compounds that possess potent immunomodulating activity including but not limited to antiviral and antitumor activity. Certain IRM compounds modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-1 and TNF (U.S. Pat. No. 6,518,265).

Certain IRM compounds are small organic molecules (e.g., molecular weight under about 1000 Daltons, preferably under about 500 Daltons, as opposed to large biological molecules such as proteins, peptides, nucleic acids, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0091491; 2004/0147543; and 2004/0176367; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, and WO 2005/079195.

Additional examples of small molecule IRM compounds include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), certain 3-β-D-ribofuranosylthiazolo[4,5-d]pyrimidine derivatives (such as those described in U.S. Publication No. 2003/0199461), and certain small molecule immuno-potentiator compounds such as those described, for example, in U.S. Patent Publication No. 2005/0136065.

Other IRM compounds include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304. Still other IRM nucleotide sequences include guanosine- and uridine-rich single-stranded RNA (ssRNA) such as those described, for example, in Heil et al., Science, vol. 303, pp. 1526-1529, Mar. 5, 2004.

Other IRM compounds include biological molecules such as aminoalkyl glucosaminide phosphates (AGPs) and are described, for example, in U.S. Pat. Nos. 6,113,918; 6,303,347; 6,525,028; and 6,649,172.

In some embodiments of the present invention, the IRM moiety may be an agonist of at least one TLR such as, for example, TLR7 or TLR8. The IRM may also in some cases be an agonist of TLR 9. In some embodiments of the present invention, the IRM compound may be a small molecule immune response modifier (e.g., molecular weight of less than about 1000 Daltons).

In some embodiments of the present invention, the IRM moiety may include a 2-aminopyridine fused to a five membered nitrogen-containing heterocyclic ring, or a 4-aminopyrimidine fused to a five membered nitrogen-containing heterocyclic ring.

IRM compounds suitable for use as the basis for the IRM moiety include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM moiety may be, or be derived from, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

Suitable IRM compounds also may include the purine derivatives, imidazoquinoline amide derivatives, benzimidazole derivatives, adenine derivatives, aminoalkyl glucosaminide phosphates, small molecule immuno-potentiator compounds, and oligonucleotide sequences described above. In some embodiments, the IRM compound may be a compound identified as an agonist of one or more TLRs such as, for example, agonists of TLR7 and/or TLR8—e.g., a TLR7-selective agonist, a TLR8-selective agonist, or a TLR7/8 agonist.

The PEG moiety may be, or be derived from, any suitable PEG polymer. In some cases, the resulting IRM-PEG complex possesses a molecular weight of at least 16 kilodaltons (kDa). In some embodiments, the resulting IRM-PEG complex may possess a molecular weight of at least 20 kDa. In other embodiments, the IRM-PEG complex has a molecular weight of at least 30 kDa.

In many embodiments, the IRM-PEG complex has a molecular weight of no greater than 500 kilodaltons (kDa). In some embodiments the IRM-PEG complex has a molecular weight of no greater than 200 kDa. In certain embodiments, the IRM-PEG complex has a molecular weight of no greater than 100 kDa, and often no greater than 50 kDa.

Various possible PEG polymers, and methods for attaching the PEG polymers to an IRM compound, are described for example, in International Patent Publication No. WO2005/110013.

Some PEG polymers may include a plurality of sites at which an IRM moiety may be attached. Thus, an IRM-PEG complex may include a plurality of IRM moieties. In such cases, the plurality of IRM moieties may be homogeneous (i.e., derived from the same IRM compound) or may be heterogeneous (i.e., derived from different IRM compounds).

An IRM-PEG complex in a thermoresponsive gel can provide active, or potentially active, IRM compound to a localized tissue region and/or tissue type, while reducing overall systemic activity of the IRM. In some cases, the IRM-PEG complex may be of a size and chemical nature to allow preferential deposition in tissues (e.g., particular tissue types or localized tissue regions) such as solid tumors. This can occur as a result of the tissue's increased vascular permeability, for example, to an IRM-PEG complex and the reduced lymphatic drainage of tumor tissues.

One or more IRM moieties can be attached to a PEG moiety through either covalent attachment or non-covalent attachment. Non-covalent attachment of an IRM moiety to a macromolecule moiety includes, for example, affinity attachment (e.g., avidin-biotin).

Representative methods for covalently attaching an IRM moiety to a PEG moiety include chemical crosslinkers, such as heterobifunctional crosslinking compounds (i.e., “linkers”) that react to form a bond between a reactive group (such as hydroxyl, amino, amido, or sulfhydryl groups) in an immune response modifier and other reactive groups (of a similar nature) in the PEG. This bond may be, for example, a peptide bond, disulfide bond, thioester bond, amide bond, thioether bond, and the like. IRM compounds can also be covalently attached to a PEG by reacting an IRM containing a reactive group directly with a polymer containing a reactive group. Methods for attaching an IRM moiety to a PEG moiety are described in detail in, for example, International Patent Publication No. WO2005/110013.

Regardless of the particular method used to couple the IRM moiety and the PEG moiety, the link may be cleaved by, for example, hydrolysis or enzymatic activity to yield free IRM compound. In reaction schemes in which the PEG moiety is attached to an IRM moiety by the formation of an amide with the 4-amino group of the IRM (e.g., Example 1) the IRM-PEG complex may provide an IRM prodrug. That is, the IRM-PEG complex may have little or no IRM activity. However, once the link between the IRM moiety and the PEG moiety is cleaved, the free IRM compound may exhibit IRM activity.

In embodiments in which the IRM-PEG complex provides an IRM prodrug, cleavage of the link between the IRM moiety and the PEG moiety may be controlled to some extent. For example, the link may be designed to be hydrolyzed in a particular biological microenvironment. The extracellular environment of tumors is known to be more acidic than the extracellular environment of normal tissues. Thus, the IRM-PEG complex may be designed as a prodrug in which the link between the IRM moiety and the PEG moiety remains intact at normal tissue extracellular pH (7.4-7.5), but is hydrolyzed in a solid tumor extracellular pH (less than 7.2). Thus, a pharmaceutical composition that includes an IRM-PEG complex and an anti-tumor antigen may be administered in the vicinity of a solid tumor. The IRM-PEG complex and antigen can infiltrate the tumor environment (e.g., by diffusion from the thermoresponsive gel carrier) where the IRM-PEG complex is cleaved to yield free IRM. This results in the co-localization of anti-tumor antigen and free IRM that can be co-delivered to immune cells in the vicinity of the tumor, thereby generating an antigen-specific, and therefore tumor-specific, immune response.

In other embodiments, the link between the IRM moiety and the PEG moiety may be designed so that the link is not cleaved unless and until the complex reaches the endosomes of an immune cell (e.g., an antigen presenting cell such as a dendritic cell).

The size and structure of the PEG moiety may influence the kinetics under which the link between the IRM moiety and the PEG moiety is cleaved. For example, a PEG moiety may include a poly-armed PEG (e.g., Example 1). The number and size of the PEG arms may influence the kinetics of enzymatic cleavage of the IRM-PEG linkage, thereby releasing free IRM. As another example, the nature of the link between the IRM moiety and the PEG moiety can impact on the rate at which the link is cleaved by hydrolysis. Amide linkages tend to be more readily hydrolyzed than carbamate linkages.

The composition also contains an antigen against which an antigen-specific immune response is desired. The antigen may be any substance that is capable of eliciting an immune response. The antigen may be, for example, a microbial antigen or a tumor antigen.

A microbial antigen as used herein is an antigen of a microorganism and includes but is not limited to antigens of viruses, bacteria, parasites, and fungi. Such antigens may include the intact organism or, alternatively, natural isolates, fragments, or derivatives thereof. A microbial antigen also may be a synthetic compound that is identical to or similar to a natural microorganism antigen and induces an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Polypeptides of bacterial pathogens include but are not limited to an iron-regulated outer membrane protein, (IROMP), an outer membrane protein (OMP), and an A-protein of Aeromonis salmonicida which causes furunculosis; p57protein of Renibacterium salmoninarum which causes bacterial kidney disease (BKD); major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius.

A tumor antigen as used herein is a compound, such as a peptide or protein, associated with a tumor or cancer cell surface and which is capable of provoking an immune response when expressed on the surface of an antigen presenting cell in the context of an MHC molecule. Tumor antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Such antigens can be isolated or prepared recombinantly or by any other means known in the art.

As used herein, tumor antigen refers to an antigen that is differentially expressed by cancer cells and can thereby be exploited in order to target cancer cells. Tumor antigens are antigens that can potentially stimulate apparently tumor-specific immune responses. Some of these antigens are encoded, although not necessarily expressed, by normal cells. These antigens can be characterized as those that are normally silent (i.e., not expressed) in normal cells, those that are expressed only at certain stages of differentiation, and those that are temporally expressed such as embryonic and fetal antigens. Other tumor antigens are encoded by mutant cellular genes, such as oncogenes (e.g., activated ras oncogene), suppressor genes (e.g., mutant p53), fusion proteins resulting from internal deletions or chromosomal translocations. Still other tumor antigens can be encoded by viral genes such as, for example, those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e. g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancers or tumors and tumor-antigens associated with such tumors (but not exclusively), include acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkins lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100 Pmel117).

Particular conditions that may be treated by administering compositions of the invention to a subject include conditions in which treatment may be mediated by an immune response against an appropriate antigen associated with the condition. Consequently, conditions that may be treated by administering a composition of the invention, including an appropriate antigen for treating the condition, include but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis camii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

The pharmaceutical compositions of the invention include the IRM-PEG complex and antigen formulated in a thermoresponsive gel. As used herein, a thermoresponsive gel formulation is a formulation that is a liquid at about 20° C., but forms a gel at warmer temperatures. For example, certain thermoresponsive gels may transition from liquid to gel at a temperature of from about 30° to about 37° C. Suitable thermoresponsive gel formulations are described, for example, in U.S. Patent Publication No. US2004/0151691.

The thermoresponsive gel may be pluronic-based or based on any suitable thermoresponsive gel polymer system. A pluronic-based thermoresponsive gel may include PEO-PPO-PEO triblock copolymers such as, for example, PLURONIC F127 (PF127) and LUTROL F127 (Poloxamer 407), both commercially available from BASF, Florham Park, N.J. LUTROL F127 is a pharmaceutical grade of PLURONIC F127 and is a PEO-PPO-PEO triblock copolymer with terminal hydroxyl groups. The percentage by weight of PEO is approximately 70% and the molecular weight calculated on the OH value is 9840 to 14,600 g/mol. Other thermoresponsive gels include PEG-PLGA-based triblock copolymers (i.e., PLGA-PEG-PLGA triblocks or PEG-PLGA-PEG triblocks) or PEG-PLGA-based diblock copolymers.

The thermoresponsive gels may be delivered into a desired localized tissue region via any suitable route, e.g., including but not limited to a subcutaneous, intradermal, intramuscular, intrathecal, intra-organ, intratumoral, intralesional, intravesicle, and intraperitoneal route of delivery. A “localized tissue region” will generally be a relatively small portion of the body, e.g., less than 10% by volume, and often less than 1% by volume. For example, depending on the size of, e.g., a solid tumor or cancerous organ, the localized tissue region will typically be on the order of no more than about 500 cm³, often less than about 100 cm³, and in many instances 10 cm³ or less. For some applications the localized tissue region will be 1 cm³ or less (e.g., for small tumor nodules, viral lesions, or vaccination sites). However, in certain instances the localized tissue region may be a particularly large region, up to several liters, for example to treat metastasized cancer within the entire peritoneal cavity (e.g., using an thermoresponsive gel to retain the IRM-PEG complex and antigen for an extended time within the peritoneal cavity). The thermoresponsive gels may be delivered using, for example, needle injection, surgical, laparoscopic, or catheter implantation, microneedle array, high-velocity particle implantation, or any other known method for introducing a preparation into a localized tissue region. Delivery to the localized tissue region may be in conjunction with image guiding techniques using, for example, ultrasound, MRI, real-time X-ray (fluoroscopy), etc.

Dosages may be figured based on the amount of IRM moiety provided by administering a given amount of the IRM-PEG/antigen/thermoresponsive gel composition. The precise amount of IRM moiety to be administered will vary according to factors known in the art including but not limited to the physical and chemical nature of the IRM moiety and the thermoresponsive gel in the composition, the amount and identity of IRM moieties provided in the IRM-PEG complex, the intended dosing regimen, the state of the subject's immune system (e.g., suppressed, compromised, stimulated), and the species to which the composition is being administered. Accordingly, it is not practical to set forth generally the amount that constitutes an amount of the composition effective for all possible applications. Those of ordinary skill in the art, however, can readily determine the appropriate amount with due consideration of such factors.

In some embodiments, the methods of the present invention include administering sufficient IRM-PEG complex to provide a dose of IRM moiety of, for example, from about 100 ng/kg to about 50 mg/kg to the subject, although in some embodiments the methods may be performed by providing the IRM moiety in a dose outside this range. In some of these embodiments, the method includes providing a dose of the IRM moiety of from about 1 μg/kg to about 5 mg/kg to the subject, for example, a dose of about 1 μg/kg, 10 μg/kg, 100 μg/kg, or 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the methods of the present invention may include administering sufficient IRM moiety to provide a dose of, for example, from about 0.01 mg/m² to about 10 mg/m².

The dosing regimen may depend at least in part on many factors known in the art including but not limited to the physical and chemical nature of the IRM moiety and the thermoresponsive gel in the composition, the amount and identity of the IRM moieties provided in the IRM-PEG complex, the amount of the composition being administered, the state of the subject's immune system (e.g., suppressed, compromised, stimulated), the method of administering the composition, and the species to which the formulation is being administered. Accordingly it is not practical to set forth generally the dosing regimen effective for all possible applications. Those of ordinary skill in the art, however, can readily determine an appropriate dosing regimen with due consideration of such factors.

In some embodiments of the invention, the composition may be administered, for example, from a one-off dose to about multiple doses per day, although in some embodiments the methods of the present invention may be performed by administering the composition at a frequency outside this range. In certain embodiments, the composition may be administered from about once per day to about once per month. In one particular embodiment, the composition may be administered once per week for six months.

The methods of the present invention may be performed on any suitable subject. Suitable subjects include but are not limited to animals such as but not limited to humans, non-human primates, poultry, fowl, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Example 1

### NHS-Activated mw 40,000 PEG

A suspension of N,N′-disuccinimidyl carbonate (18.5 g, 72 mmol) in 300 mL of CH₂Cl₂ was added to a solution of 8 ARM PEG MW 40,000 (Nektar, Cat. No. 0J000T08) in 500 mL of CH₂Cl₂. N,N-Dimethylaminopyridine (8.8 g, 72 mmol) was then added and the mixture was stirred under N₂ for 3 days. The reaction mixture was then concentrated under reduced pressure and then concentrated from 200 mL of acetone to give a syrup. The syrup was treated with 300 mL of acetone and the stirred mixture was warmed until it became homogenous. Diethyl ether (900 mL) was then slowly added and then the mixture was placed in an ice bath. Stirring was continued for 10 minutes as a white solid formed. The solid was isolated by filtration and dried with suction. The solid was again subjected to the acetone/diethyl ether precipitation process two more times to give a white solid. The solid was rinsed with 500 mL of diethyl ether and dried with suction to give a white powder. The resulting material was transferred to a flask and dried vacuum to give 71.6 g of the desired product.

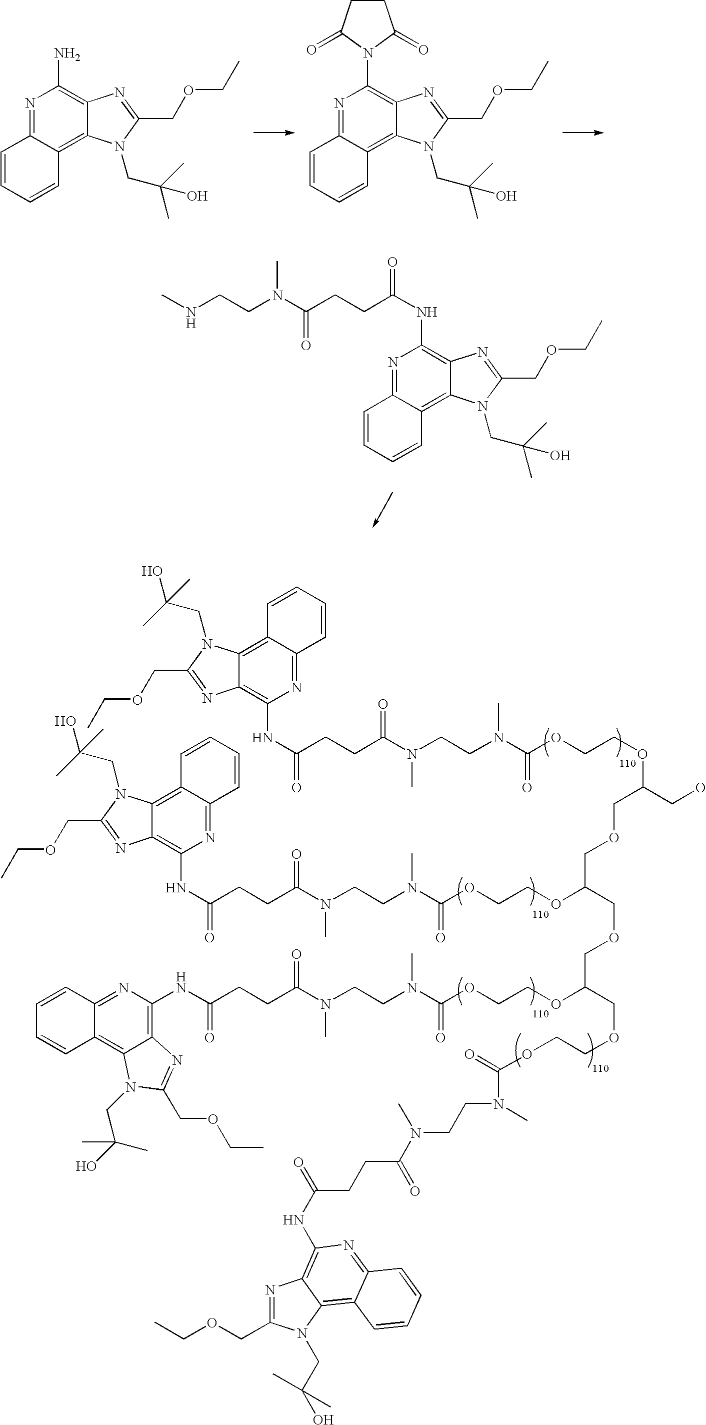

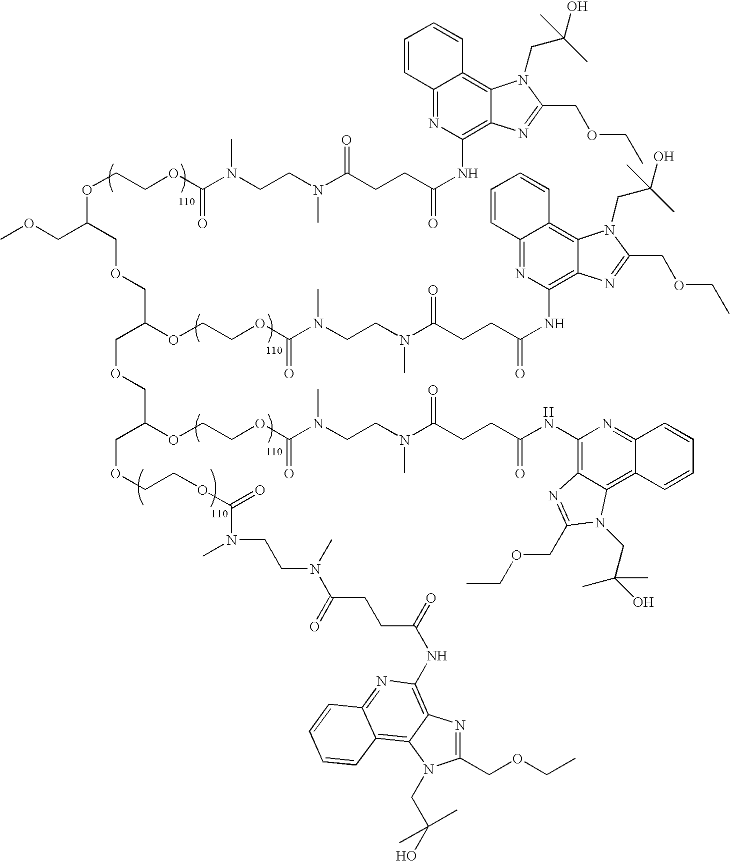

### Part A

A 1 L-round bottom flask, equipped with a Dean-Stark trap, was charged with 4-amino-α,α-dimethyl-2-ethoxymethyl-1H-imidazo[4,5-c]quinolin-1-ethanol (i.e. 1-[4-amino-2-(ethoxymethyl)-1H-imidazo[4,5-c]quinolin-1-yl]-2-methylpropan-2-ol (31.4 g, 100 mmol), synthesis of which is described, for example, at U.S. Pat. No. 5,389,640, Example 99. Anhydrous toluene (500 mL) was added followed by succinic anhydride (10.0 g, 100 mmol) and the mixture was heated to reflux for 24 hours. Another 10.0 g succinic anhydride was added to the reaction mixture and heating was continued for 2 days. The reaction was still not complete so an additional 10.0 g of succinic anhydride was added and heating was continued for 4 days. The reaction mixture was then cooled and filtered to give a white powder. The white powder was stirred in refluxing methanol (300 mL), cooled and filtered to give a white solid. The treatment with refluxing methanol was repeated two more times to give a white powder that was dried by suction and then under vacuum at 100° C. to give 29.6 g of 1-[2-(ethoxymethyl)-1-(2-hydroxy-2-methylpropyl)-1H-imidazo[4,5-c]quinolin-4-yl]pyrrolidine-2,5-dione.

### Part B

A stirred suspension of 1-[2-(ethoxymethyl)-1-(2-hydroxy-2-methylpropyl)-1H-imidazo[4,5-c]quinolin-4-yl]pyrrolidine-2,5-dione (7.92 g, 20.0 mmol) in 100 mL of tetrahydrofuran (THF) was treated with N,N′-dimethylethylenediamine (10.6 mL, 100 mmol) and 4-dimethylaminopyridine (DMAP, 244 mg, 2.0 mmol) and the mixture was heated to 56° C. under an atmosphere of N₂. After stirring overnight, the reaction mixture was concentrated under reduced pressure to give a syrup. The syrup was dissolved in 100 mL of CH₂Cl₂ and washed with H₂O (2×50 mL) and brine (50 mL). The organic portion was dried over Na₂SO₄, filtered and concentrated under reduced pressure to give a white foam. Column chromatography (SiO2 50-100% CMTEA/CHCl₃ (CMTEA=80:18:2 CHCl₃/MeOH/Et₃N)) gave the desired material as a white foam. The foam was dried under vacuum overnight to give N¹-[2-(ethoxymethyl)-1-(2-hydroxy-2-methylpropyl)-1H-imidazo[4,5-c]quinolin-4-yl]-N⁴-methyl-N⁴-[2-(methylamino)ethyl]succinamide (3.76 g) as a white solid.

### Part C

Activated PEG NHS ester (24.7 g, 0.60 mmol) was dissolved in 200 mL of anhydrous CH₂Cl₂ and treated with N¹-[2-(ethoxymethyl)-1-(2-hydroxy-2-methylpropyl)-1H-imidazo[4,5-c]quinolin-4-yl]-N⁴-methyl-N⁴-[2-(methylamino)ethyl]succinamide (3.76 g, 7.78 mmol) and DMAP (146 mg, 1.20 mmol). After stirring under N₂ for 2 days the reaction mixture was concentrated under reduced pressure to give a syrup. The syrup was treated with 100 mL of acetone and the stirred mixture was warmed until it became homogenous. Diethyl ether (300 mL) was then slowly added and then the mixture was placed in an ice bath. Stirring was continued for 10 minutes as a white solid formed. The solid was isolated by filtration and dried with suction. The solid was again subjected to the acetone/diethyl ether precipitation process two more times to give a white solid. The solid was then dissolved with 150 mL of hot 2-propanol and then cooled to give a white solid. The precipitation from 2-propanol was repeated and the final product was dried under vacuum for 2 days to give a white powder (24.4 g).

### Example 2

A 0.1 mg/mL ovalbumin (OVA, Pierce Biotech, Rockford, Ill.) solution was made in PBS, pH 7.4. An OVA solution containing the final product from Example 1 (IRM-PEG) was made by dissolving 104 mg of the IRM-PEG into 10 mL of the OVA solution to get an IRM-PEG OVA solution equivalent to 0.5 mg/mL IRM and 0.1 mg/mL OVA. Serial dilutions of 1:10 were performed using the IRM-PEG OVA solution and the 0.1 mg/mL OVA solution to prepare additional solutions with IRM equivalences of 0.05, 0.005, 0.0005 mg/mL IRM in 0.1 mg/mL OVA.

An OVA solution containing 20% (w:w) PF127 (BASF, Florham Park, N.J.) was made by adding 3 grams of PF127 to 12 grams of the 0.1 mg/mL OVA solution and refrigerated overnight to dissolve the PF127. An hour or less before dosing, 104 mg of IRM-PEG was dissolved in 10 mL of the 0.1 mg/mL OVA containing 20% PF127. Serial dilutions of 1:10 were performed using the IRM-PEG containing OVA-PF127 solution and the 0.1 mg/mL OVA solution to prepare additional solutions with IRM equivalences of 0.05, 0.005, 0.0005 mg/mL IRM in 0.1 mg/mL OVA.

### Example 3

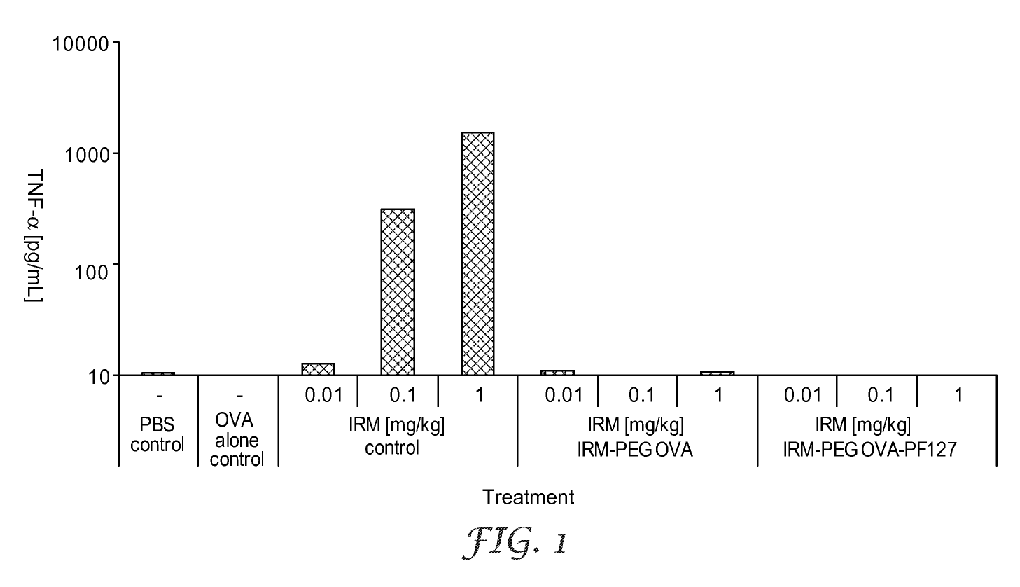

Female 4 to 6 week old C57BL/6 mice (Charles River Laboratory, Wilmington, Mass.) were injected intramuscularly in the left lower leg with 50 μL of the OVA solution; IRM-PEG OVA solutions; or IRM-PEG OVA-PF127 solutions, all made in Example 2; 0.01, 0.1, or 1.0 mg/kg of 4-amino-α,α-dimethyl-2-ethoxymethyl-1H-imidazo[4,5-c]quinolin-1-ethanol (IRM); or PBS. Blood was collected one-hour post dose by cardiac puncture and the serum was analyzed for mouse TNF-α by ELISA (Biosource, Carmarillo, Calif.). Results are shown in FIG. 1.

### Example 4

4×10⁶ chicken ovalbumin-specific OT-1.PL (Thy-1.1⁺) lymphocytes per mouse were adoptively transferred into syngeneic 4-6 week old female C57BL/6 mice (Charles River Laboratories, Wilmington, Mass.).

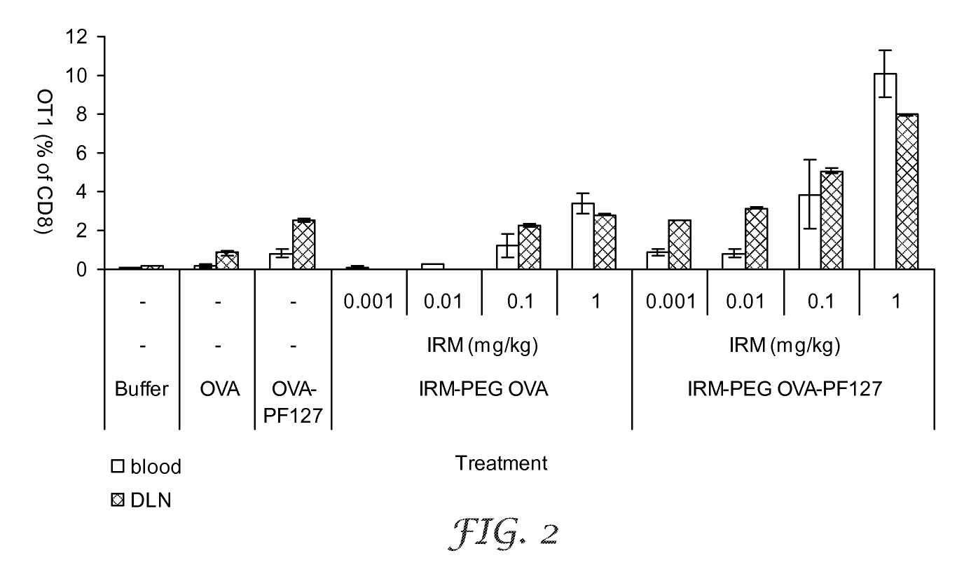

Two days later, the mice were immunized intramuscularly in each of the lower legs with 50 μL of the treatments (n=3 mice per treatment) described in Example 3. Four days after immunization, mice were bled by cardiac puncture and the popliteal lymph nodes were removed and homogenized into a single cell suspension. Lymphocytes from the suspension were stained, in triplicate, with mouse anti-Fc, FITC-labeled mouse anti-CD8 (BD Pharmigen), APC-labeled mouse anti-Thy1.1 (BD Pharmigen), and PerCP-labeled mouse anti-CD3 (BD Pharmigen). Cells were incubated for 30 minutes at room temperature, washed with Flow Cytometry Staining Buffer (Biosource), resuspended in Cytofix (BD Pharmigen) for 10 minutes, washed with Flow Cytometry Staining Buffer, filtered, and analyzed on a FACSCaliber (Becton, Dickinson, and Co., San Jose, Calif.). CD8⁺ Thy1.1⁺ T cells were recorded as a percentage of CD8⁺ T-cells. Results are found in FIG. 2.

### Example 5

A pharmacokinetic study to determine the systemic TNF-a cytokine levels as a function of time (1, 2, 6, and 24 hours post dosing) was conducted in mice comparing intramuscular formulations containing free IRM+OVA solution, IRM-PEG+OVA solution, and IRM-PEG+OVA in 20% (w/w) LUTROL F127 gel. Two different doses of IRM were used, 1 mg/Kg and 0.1 mg/Kg, corresponding to formulation concentrations of 0.5 and 0.05 mg/mL. The OVA concentration was kept constant at 0.1 mg/mL. At the 0.1 mg/Kg dose the IRM-PEG gel formulation showed a substantial reduction in serum TNF-α concentration when compared to free IRM. At the 1 mg/Kg dose the IRM-PEG gel formulation showed a substantial reduction in serum TNF-a concentration when compared to free IRM and to IRM-PEG.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A pharmaceutical composition comprising:
an IRM-PEG complex and an antigen, formulated together in a thermoresponsive gel.

2. The pharmaceutical composition of claim 1 wherein the IRM-PEG complex is in the form of an IRM prodrug.

3. The pharmaceutical composition of claim 1 wherein the IRM-PEG complex comprises an IRM portion that comprises, or is derived from, an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

4. The pharmaceutical composition of claim 1 wherein the thermoresponsive gel comprises PEO-PPO-PEO triblock copolymers.

5. The pharmaceutical composition of claim 1 wherein the thermoresponsive gel comprises PF127.

6. The pharmaceutical composition of claim 1 wherein the thermoresponsive gel comprises PEG-PLGA-based triblock copolymers.

7. The pharmaceutical composition of claim 6 wherein the thermoresponsive gel comprises PEG-PLGA-PEG triblocks.

8. The pharmaceutical composition of claim 6 wherein the thermoresponsive gel comprises PLGA-PEG-PLGA triblocks.

9. The pharmaceutical composition of claim 1 wherein the thermoresponsive gel comprises PEG-PLGA diblocks that are liquid at about 20° C. and form a gel at from about 30° C. to about 37° C.

10. The pharmaceutical composition of claim 1 wherein the antigen comprises a tumor antigen.

11. The pharmaceutical composition of claim 1 wherein the antigen comprises a bacterial antigen.

12. The pharmaceutical composition of claim 1 wherein the antigen comprises a viral antigen.

13. A method of eliciting an antigen-specific immune response in a subject, the method comprising:
administering to the subject a pharmaceutical composition comprising an IRM-PEG complex and an antigen, formulated together in a thermoresponsive gel, in an amount effective to generate an immune response in the subject against the antigen.

14. A method of treating a condition in a subject, the method comprising:
administering to the subject a pharmaceutical composition comprising an IRM-PEG complex and an antigen, formulated together in a thermoresponsive gel, in an amount effective to ameliorate at least one symptom or clinical sign of the condition.

15. The use of an IRM compound for the manufacture of a pharmaceutical composition comprising an IRM-PEG complex and an antigen, formulated together in a thermoresponsive gel.
